(19) United States
(12) Patent Application Publication
(10) Pub. No.: US 2026/0076535 A1
(43) Pub. Date: Mar. 19, 2026

(54) BOUGIE INTRODUCER WITH VIDEO IMAGING

(71) Applicant: Acera LLC, Beverly, MA (US)

(72) Inventors: Michael Cook, Salem, MA (US); Thomas V. Root, Beverly, MA (US)

(73) Assignee: Acera LLC, Beverly, MA (US)

(21) Appl. No.: 19/307,675

(22) Filed: Aug. 22, 2025

Related U.S. Application Data
(60) Provisional application No. 63/686,011, filed on Aug. 22, 2024.
(60) Provisional application No. 63/782,327, filed on Apr. 2, 2025.

(51) Int. Cl. A61B 1/267; A61B 1/00; A61B 1/04; A61B 1/06

(52) U.S. Cl. CPC A61B 1/267; A61B 1/00032; A61B 1/00045; A61B 1/00101; A61B 1/00103; A61B 1/00147; A61B 1/042; A61B 1/0661

(57) ABSTRACT

An ETT guide introducer (bougie) with video imaging, which comprises a body part including a hollow inner passageway extending to a distal section of the body part; and a video camera system including a housing mounted in the tip. The camera system further includes a camera and a light source configured to be inserted in the head section. Additionally, a method for external position visualization via transillumination using red light, a capability to deliver oxygen near the distal end, and the ability to inflate a balloon for widening an anatomical lumen.

### RELATED APPLICATIONS

The present application claims priority to U.S. Provisional Application No. 63/686,011 filed on Aug. 22, 2024 and U.S. Provisional Application No. 63/782,327 filed on Apr. 2, 2025, each of which is herein incorporated by reference in its entirety.

### TECHNICAL FIELD

The present disclosure relates generally to devices for providing access to and/or the visualization and/or the strictureplasty of, anatomical lumens, such as devices for guiding endotracheal tubes and more specifically bougie introducers utilized with endotracheal tubes.

### BACKGROUND

It is estimated that over 15 million endotracheal intubations are performed each year in the USA alone. These patients need “airway management” as is best afforded by placement of an Endotracheal Tube (ETT) to facilitate breathing. Often, only an ETT can provide an “assured protected airway” due to some trauma or impaired spontaneous breathing.

When an ETT is inserted into a patient, the ETT usually causes some discomfort and stress, particularly as pressure is applied to soft tissue or if the ETT is accidentally inserted into the esophagus. The patient may experience a gag-reflex, and any friction with the device may harm or choke the patient.

To address some of the above issues, sometimes an ETT guide is utilized. In some cases, the ETT guide may be a bougie. The ETT guide may facilitate insertion of the ETT. More specifically, because the ETT guide may be smaller than the ETT, firm, or easily manipulated, it may be passed into the trachea first and then used as a guide to “railroad” the ETT into place. While some ETT guides are straight, some other versions have a bent “coude”tip to assist in navigating around corners. Moreover, most manufacturers offer bougies in a variety of sizes (diameters, in French size) to accommodate patients large and small. Bougies and ET Tubes are typically marked along the length to indicate depth of penetration, which lets the doctor know how deep they are in the body. Moreover, a water-based lubricant may be used with an endotracheal tube (ETT) introducer, commonly known as a “gum elastic bougie”.

In some cases, endotracheal intubation may be done with a bladed laryngoscope or video laryngoscope. This procedure often requires a second assistant holding the scope to help in visualizing the trachea. Moreover, this procedure is often very painful to the patient because of which drugs may be administered to alleviate sympathetic response and airway reflexes. The patient may be preoxygenated for 3 minutes before beginning and then given an anesthetic (for example, by IV), such as propofol or fentanyl, a hypnotic. This may cause loss of consciousness and respiratory drive. A paralytic may then be administered while ventilation is maintained mechanically. The patient's breathing may thus become the doctor's responsibility. The patient's lips may get pinched by, or teeth may contact, the scope's blade unless care is taken. Further high skills are needed for insertion in the mouth. Positioning of the patient's head and neck are important, and a “jaw thrust” is typically needed for access to the airway. Lifting the scope's blade while inserted however may sometimes break the patient's teeth. Once the vocal cords are visible, the ETT may be inserted past them into the trachea and the cuff inflated. The cuff may be needed to prevent aspiration and to fill the space between the ETT outer diameter (OD) and the trachea, for securing the ETT's position.

Some practitioners suggest that a bougie should always be used during an endotracheal intubation (ETI) and not just for difficult cases. In some cases, if the glottic opening is not visible, correct placement of an ETT guide into the trachea is blindly done by feel. Typically, tactile feedback is felt as “clicks” when the bougie slides over tracheal rings. If there are no clicks felt, then the bougie is likely in the esophagus. Advancing the bougie past the cricoid ring may let it penetrate the trachea and it may then rotate as it enters the main stem bronchus, stopping only when the smaller delicate bronchi are reached. Even a small amount of force applied here may risk perforating the tissue. Sometimes, the practitioner may feel for “hold-up” to determine that the position is correct. Such practices may cause harm to the patient. In particular, the bougie, because of its stiffness, may cause damage or perforation of the trachea, bronchi, and potentially the esophagus.

Additional problems may arise because the endotracheal procedure may need to be performed rapidly in an ED, OR, ICU, or even in a life/death situation such as at the scene of an accident by a first responder. The patient may be unconscious, and may be a young child or an elderly dementia patient, who cannot understand or cooperate. These difficult intubations take longer to perform and are more likely to require multiple attempts. Blocking the patient's airway may cause stress and may increase the likelihood of a stroke or heart attack. Similar problems may arise when the procedure is performed on newborn babies, also known as neonatal resuscitation in the delivery room. Approximately 1% of newborn babies need this intervention.

Further, a medical professional may need to visualize other anatomical lumens and/or insert devices, such as, catheters, into such lumens and/or widen strictures or dislodge obstructions in the lumens.

### SUMMARY

Further understanding of various aspects of the embodiments may be obtained by reference to the following detailed description in conjunction with the associated drawings, which are described briefly below.

In one aspect, an intraluminal navigation device is disclosed, which includes an intraluminal segment extending from a proximal end to a distal end and configured to be inserted into a body lumen, and a tip-mounted assembly that is coupled to the distal end of the intraluminal segment. The tip-mounted assembly includes a surface having at least one opening, at least one light source configured to generate illumination light to be transmitted through said at least one opening, and at least one video camera positioned to receive light entering the tip-mounted assembly through said at least one opening and configured to generate video images for guiding insertion of said intraluminal segment into said body lumen.

In various embodiments, the intraluminal segment includes a recess (e.g., a cavity) at the distal end thereof for receiving and housing the tip-mounted assembly.

In various embodiments, the tip-mounted assembly includes a housing having a first recess in which said video camera is disposed and a second recess in which said at least one light source is disposed.

In various embodiments, the intraluminal segment is configured for insertion into a subject's trachea.

In various embodiments, the at least one light source includes a plurality of light sources surrounding said video camera.

In various embodiments, the video camera is configured to transmit the video images wirelessly to a display for presentation to a user.

In various embodiments, the intraluminal segment can be formed of a polymeric material.

In various embodiments, the device can be a single-use and disposable device.

In various embodiments, the intraluminal segment can be substantially tubular. In some such embodiments, the intraluminal segment can have an outer diameter equal to or less than about 6.33 mm, e.g., in a range of about 4 mm to about 5 mm (19Fr=6.33 mm OD max, 15Fr=5 mm typical, 12Fr=4 mm OD min), and an inner diameter in a range of about 1.5 mm to about 4.2 mm, all by way of example.

In various embodiments, the device can include a compartment for housing one or more batteries for providing electrical power to the light source(s) and the video camera.

In various embodiments, the intraluminal segment can be hollow. In some such embodiments, the intraluminal segment can include an opening. Further, in some such embodiments, the device can further include an inflatable cuff that is coupled to said intraluminal segment upstream of said camera system and in fluid communication with the opening formed in the intraluminal segment as well as a mechanism coupled to the proximal end of the intraluminal segment for inflating the inflatable cuff via said hollow lumen and said opening in said intraluminal segment.

In various embodiments, the tip-mounted assembly further includes at least one red light source that is configured to generate red light and is positioned such that the red light is transmitted through said opening formed in the intraluminal segment with at least a portion of the transmitted red light passing through a wall of said body lumen and surrounding tissue so as to be externally detectable.

Some embodiments relate to a device for assisting insertion of an endotracheal tube into a patient, the device including: a tubular element configured for guiding the endotracheal tube into the patient's trachea, the tubular element extending from a proximal end to a distal end and configured for insertion into the patient's trachea, and a camera system and one or more light sources, such as LED(s), embedded in the distal tip of the tubular element for providing video images for guiding the insertion of the tubular element into the patient's trachea.

Some embodiments relate to a device, wherein the tubular element includes a recess at the distal end thereof for receiving the camera system and the light sources (e.g., LED(s)).

Some embodiments relate to a device, wherein the camera system includes: a housing having two recesses (e.g., in the form of cavities or grooves), a video camera positioned in one of the recesses, and a light source positioned in the other recess.

Some embodiments relate to a video bougie including: a body part (herein also referred to as an intraluminal segment) defining a bougie recess (e.g., a groove) extending to a distal section of the body part; and a camera system including: a housing having a head section and an arm section, the arm section being configured for insertion into the bougie's inner recess (herein also referred to as inner diameter); a camera configured to be inserted in a cavity in the head section; and a light source configured to be inserted in or near the head section.

Some embodiments relate to a video bougie, wherein the head section is configured to protrude from the distal section of the body part.

Some embodiments relate to a video bougie, wherein the body part further defines a first hole for injection of epoxy in a peripheral recess (e.g., a peripheral groove) of the arm section of the camera housing, locking the parts together.

Some embodiments relate to a video bougie, wherein the body part further defines a second hole for injection of epoxy into the bougie recess behind the arm section of the camera system, which seals and encases wires and connections.

Some embodiments relate to a bougie camera system including: a housing having a head section and an arm section, the arm section being configured for insertion into a hollow lumen in a tubular bougie; a camera configured to be inserted in the head section; and a light source configured to be inserted in the head section adjacent to the camera.

In a related aspect, a single-use, disposable intraluminal navigation device is disclosed, which includes a single-use, disposable body part (herein also referred to as an intraluminal segment) extending from a proximal end to a distal end and configured to be inserted into an anatomical lumen, and a single-use, disposable camera system that is positioned at the distal end of the body part. The camera system can include at least one light source, such as an LED, for illuminating the lumen and at least one imaging sensor for generating video image data.

In various embodiments, such a single-use, disposable device can include multiple light sources, such as LEDs, that are positioned around the imaging sensor. By way of example, the light sources can include a plurality of LEDs that can generate light of a variety of colors, but typically a white LED is used for the visualization of the lumen.

In various embodiments, the body part of the single-use, disposable device can be formed from a polymeric material, such as those described herein. Further, in various embodiments, the body part of the bougie can have a substantially tubular shape with an outer diameter equal to or less than about 6.33 mm, e.g., in a range of about 2 mm to about 6.33 mm. In some such embodiments, the inner diameter of a hollow lumen (herein also referred to as a channel) of the bougie can be in a range of about 1 mm to about 4.2 mm, by way of example.

In various embodiments, the bougie can include a compartment for housing one or more batteries, which can be electrically connected, via a switch, to the camera system, including its light source and its imaging sensor. In some embodiments, an external tab coupled to the outer surface of the compartment can be removed to establish an electrical connection between the one or more batteries and the camera system.

In a related aspect, a single-use, disposable device is disclosed, which includes an elongated body part (herein also referred to as an elongated intraluminal segment) extending from a proximal end to a distal end and configured to be inserted into an anatomical lumen, a camera system located at the distal end of the elongated body part, a handle attached to the proximal end of the body part, said handle providing a compartment for housing control circuitry and one or more batteries for supplying electrical power to the camera system.

In some embodiments, the single-use, disposable device can be powered via a mobile device, such as a mobile phone.

In various embodiments, the camera system of the single-use, disposable device can be configured to transmit video images captured by its imaging sensor to a video display (herein also referred to as video monitor) for presentation to a user. The video monitor can be separate from, and not mechanically coupled to, the single-use, disposable device.

In a related aspect, a single use, disposable bougie is disclosed, which includes a body part (herein also referred to as an intraluminal segment) extending from a proximal end to a distal end and providing a hollow lumen and a camera system located in the distal end of the body part and at least partially disposed within said hollow lumen, where the camera system includes a light source and an imaging sensor.

An opening formed in the hollow body part fluidly couples the lumen of the body part to an inner volume of an inflatable cuff coupled to the body part upstream of said camera system. A mechanism, such as a syringe, coupled to the proximal end of the body part can be used to inflate the cuff via the hollow lumen of the body part and its aforementioned opening.

In a related aspect, a method of visualizing an anatomical lumen is disclosed, which includes inserting a single-use, disposable device into a body lumen, where the device is configured to illuminate and generate video images of the lumen. The device includes a single-use, disposable body part (herein also referred to as an intraluminal segment) extending from a proximal end to a distal end and configured for partial insertion into the lumen with a camera system located in said distal end, said camera system including at least one light source, such as at least one LED, for illuminating the anatomical lumen and at least one imaging sensor for generating video images of the lumen, and utilizing the video images generated by said camera system to advance the body part through the anatomical lumen.

In a related aspect, a single-use, disposable device is disclosed, which includes an intraluminal segment extending from a proximal end to a distal end and configured to be inserted into a body lumen. A tip-mounted assembly is located in the distal end of the intraluminal segment, where the tip-mounted assembly includes: an outer surface having at least one opening, at least one light source generating illumination light to be transmitted through said at least one opening, at least one video camera positioned to receive light entering the tip-mounted assembly via said at least one opening, and at least one red light source generating red light and positioned such that the red light is transmitted through said at least one opening with at least a portion of the transmitted red light passing through a wall of the lumen and surrounding tissue to be detected external to the patient.

In various embodiments, the at least one opening can include a central opening for the camera and a plurality of peripheral openings through which red and white light may pass while the at least one red light provides for transillumination of the lumen and the surrounding tissue to aid in visually locating the device external to the patients body

In various embodiments, any of the illumination light source(s) and the red light source(s) can be an LED. In some such embodiments, the red LED(s) can generate light at a wavelength in a range of about 600 nm to about 700 nm, and preferably at a wavelength in a range of about 660 nm to about 680 nm, with an intensity in a range of about 3 lumens to about 50 lumens. In general, the positioning and the intensity of the red light source are selected so as to provide transillumination of the lumen and its surrounding tissue such that at least a portion of the red light is detectable external to the patient's body. The externally detected light can be employed for safe insertion and guidance of the transluminal segment through a body lumen, for example by showing that the device is in the trachea and not accidentally inserted into the esophagus

In a related aspect, a single-use, disposable device is disclosed, which includes an intraluminal segment extending from a proximal end to a distal end and configured to be inserted into a body lumen, said intraluminal segment comprising a light-transmissive window formed in a wall thereof. The single-use, disposable device further includes at least one red light source coupled to said intraluminal segment such that light generated thereby can pass through said light-transmissive window for providing transillumination of at least a portion of the wall of the body lumen and at least a portion of the surrounding tissue. A tip-mounted assembly is coupled to the distal end of the intraluminal segment. The tip-mounted assembly includes an outer surface having at least one opening, at least one light source generating illumination light to be transmitted through said at least one opening, and at least one video camera positioned to receive light entering the tip-mounted assembly via said at least one opening.

Further understanding of various aspects of the present teachings can be obtained with reference to the following detailed description in conjunction with the associated drawings, which are described briefly below.

### BRIEF DESCRIPTION OF THE DRAWINGS

The drawings are not necessarily to scale or exhaustive. Instead, emphasis is generally placed upon illustrating the principles of the embodiments described herein. The accompanying drawings, which are incorporated in this specification and constitute a part of it, illustrate several embodiments consistent with the disclosure. Together with the description, the drawings serve to explain the principles of the disclosure.

In the drawings:

FIGS. 1A-1D show four different views of a coude tip (bent tip) of a video bougie 100 and its parts according to some embodiments.

FIG. 1E is a schematic view of a video bougie according to another embodiment.

FIG. 2 shows a functional block diagram of a camera and its wiring and connector according to some embodiments (although the wire cable is shown with four wires, additional wires may be present, e.g., 14 wires, for connecting the camera to a connector (e.g., USB-C or similar connector type) as well as for powering the LEDs.

FIG. 3A shows the wire and connector extending beyond the proximal end of the video bougie according to some embodiments. These wires may extend several feet beyond the proximal end of the bougie itself.

FIG. 3B shows an embodiment of a video bougie according to the present teachings, which is powered by a plurality of batteries housed in a compartment of a handle of the bougie. The PCB (3010) shown would provide a camera controller function and wireless connectivity via Bluetooth or WiFi.

FIG. 4A shows an Endotracheal Tube (ETT) 400 according to some embodiments.

FIG. 4B shows an endotracheal tube (ETT) with an inflatable cuff, often used to stabilize its position after installation and prevent aspiration of fluids into the patients lungs.

FIG. 5A schematically shows a video bougie according to an embodiment in communication with a video display (monitor or tablet).

FIG. 5B schematically shows a video bougie according to an embodiment in communication with a mobile device (e.g., a mobile phone).

FIG. 6 schematically shows a video bougie according to an embodiment that allows, in addition to visualizing an anatomical lumen, the administration of oxygen to a patient.

FIG. 7A schematically depicts a video bougie according to an embodiment, which includes an inflatable cuff (herein also referred to as an inflatable balloon) that is coupled to its body part.

FIG. 7B schematically shows a mechanism for inflating the balloon associated with the video bougie depicted in FIG. 7A,

FIG. 8 schematically depicts a video bougie according to an embodiment having a straight tubular intraluminal segment configured to be inserted into a body lumen.

FIG. 9A is a partial schematic depiction of a video bougie according to an embodiment that includes a tip-mounted assembly to which a camera and a plurality of LEDs are coupled, where some of the LEDs generate white illumination light while the other LEDs generate red transillumination light,

FIG. 9B is another schematic view of the video bougie shown in FIG. 9A. Three of the 6 LEDs shown are white and 3 are red, where the red LEDs are to be independently controlled (on/off) from the white.

FIG. 10A is a schematic view of a video bougie according to an embodiment, which includes, in addition to a camera system at its distal end, a plurality of red LEDs for emitting light from the sides of the coude tip for transilluminating its position.

FIG. 10B is a partial schematic view of the video bougie shown in FIG. 10A.

FIG. 10C is a partial side schematic view of the video bougie illustrated in FIG. 10A.

FIG. 10D is a partial schematic top view of the video bougie illustrated in FIG. 10A, providing a clearer view of the red LEDs and the respective windows through which the red light is transmitted. The flexible PCB allows installation with the LEDs partially protruding into the openings, locking them in place. A clear epoxy can be used to seal the LEDs in place. UV-curable clear epoxy may be used.

FIG. 10E schematically depicts the red LEDs of the video bougie shown FIG. 10A, where the red LEDs are mounted to a flexible printed circuit board (PCB), and demonstrates how 4 LEDs could be connected in parallel.

### DETAILED DESCRIPTION

The following detailed description refers to the accompanying drawings. The same or similar reference numbers may have been used in the drawings or in the description to refer to the same or similar parts. Also, similarly named elements may perform similar functions and may be similarly designed, unless specified otherwise. Details are set forth to provide an understanding of the exemplary embodiments. Embodiments, e.g., alternative embodiments, may be practiced without some of these details. In other instances, well known techniques, procedures, and components have not been described in detail to avoid obscuring the described embodiments.

To address the above listed multiple problems in the existing technology and procedures, some embodiments utilize an ETT guide introducer with video imaging for assisting with the placement of an Endotreacheal Tube (ETT) into a patient's airway. The use of this device may simplify the procedure and save time and money while providing better patient care. This could become the standard of care for patients who need an ETT. In some embodiments, the ETT guide introducer may be a video bougie, that is, a bougie that, among other things, has been enhanced by incorporating a camera and/or a light source. In the following detailed description, to facilitate the description and without loss of generality, the terms bougie and video bougie may be used to represent the more general cases in which other types of ETT guide introducers are used according to other embodiments.

By way of example, the term “bougie,” as used herein, refers to an instrument having an intraluminal segment that can be inserted into or through a body passageway (lumen), such as, trachea, esophagus, or urethra, for visualization of the lumen, guidance of other instruments through the lumen, widening of strictures, or dislodgement of obstructions within the lumen, among other applications.

The term “red light source,” as used herein, refers to a light source that can generate light at a wavelength in a range of about 600 nm to about 700 nm, and preferably at a wavelength in a range of about 660 nm to about 680 nm.

The term “about,” as used herein, indicates a variation of at most 10% around a numerical value.

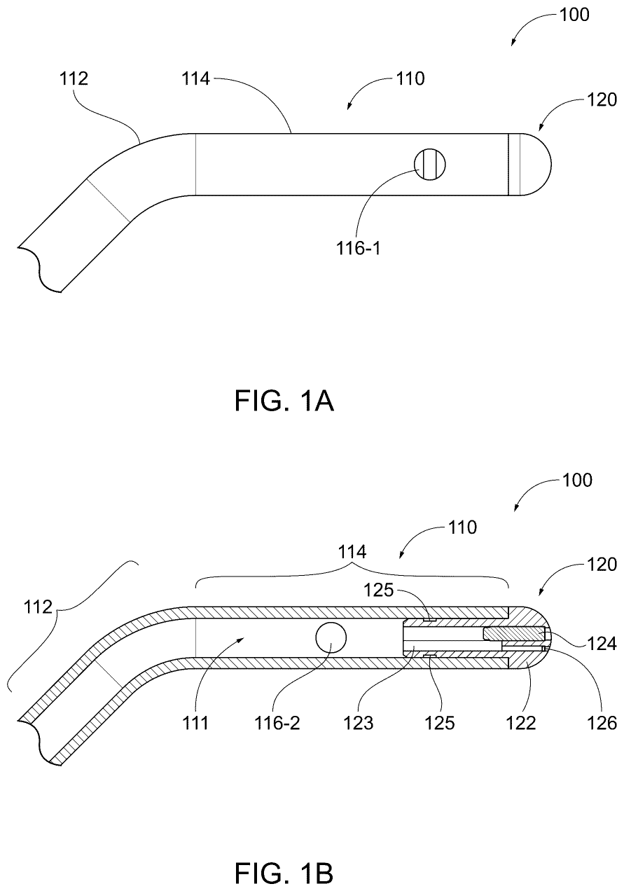

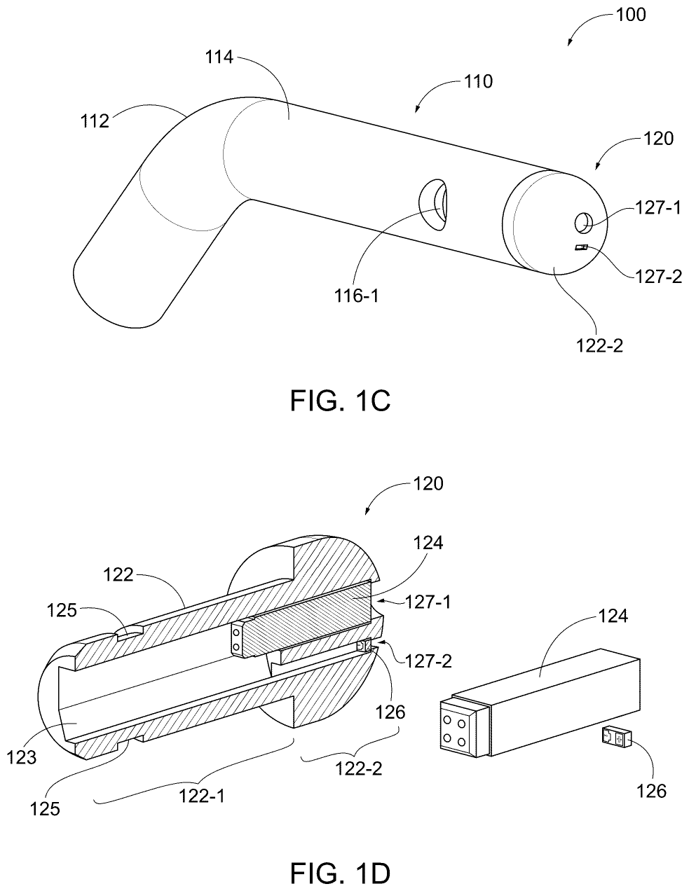

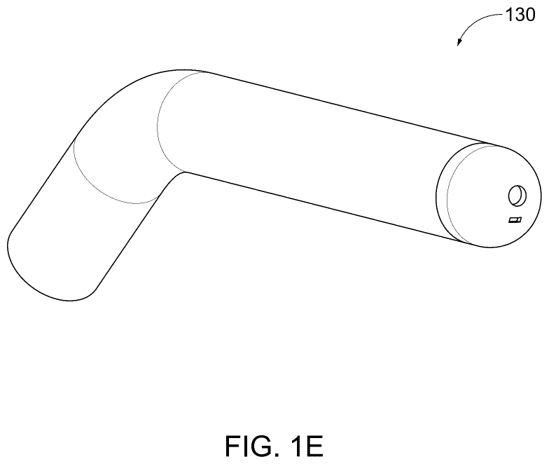

FIGS. 1A-1D show four different views of a coude tip (bent tip) of a video bougie 100 and its parts according to some embodiments. More specifically, FIG. 1A shows a side view, FIG. 1B shows a cross-sectional side view, and FIG. 1C shows an ISO view (from a distal tip looking toward a bend section, both defined further below) of video bougie 100. Further, FIG. 1D shows a cross-sectional view of a camera system 120 installed in video bougie 100 according to some embodiments.

Video bougie 100 includes a body part (intraluminal segment) 110 forming its proximal section and a camera system 120 forming its distal section. Body part 110 extends from a proximal end (not shown in the figures) to a distal end from which camera system 120 protrudes. In various embodiments, video bougie 100 may have different outer diameter sizes to fit inside different types of tubes. Video bougie 100 may, for example, have an outer diameter of 5 mm equivalent to a size 15-Fr tube. Other “French” sizes such as 5, 6, 8, 10, 11, 12, 14, and 19-Fr are considered candidates for the video bougie also and range from 1.7 mm to 6.3 mm OD.

In various embodiments, the body part 110 may be formed using one or more polymeric materials. Some examples of such polymeric materials include, without limitation, polyurethane, polyester, polyether block amide, polyvinyl chloride, polyethylene, low and high polyethylene blends, among others. Medical Grade Polyethylene (PE) is a typical latex free and flexible material. In some embodiments, the body part 110 can be formed of two or more different polymeric materials so as to impart desired flexibility and durability to the video bougie. By way of example, and without limitation, a video bougie can include an inner polyester core and an outer resin coating. In some embodiments, at least a portion of the bougie may be formed of a polymer that can undergo a plastic deformation, e.g., to allow the video bougie to exhibit a bent tip.

Body part 110 includes a bend section 112, and a distal section 114. In video bougie 100, bend section 112 has a bend angle of around 45°. Distal section 114, on the other hand, is straight.

As shown in FIG. 1B, video bougie 100 includes an interior bougie channel 111 (herein also referred to as an inner channel or a lumen 111). Bougie inner channel 111 may span all or some part of the interior of video bougie 100. In particular, inner channel 111 may span the entirety of distal section 114 to provide a housing for the insertion of camera system 120. In some embodiments, the internal diameter of bougie inner channel 111 may be around 3 mm, for example 3.2 mm.

Moreover, distal section 114 includes a first hole 116-1 and a second hole 116-2. First hole 116-1 and second hole 116-2 each penetrate the outer shell of body part 110 and provide an opening to different locations of bougie inner channel 111. More specifically, first hole 116-1 may be located at a position over a groove 125 on the outer periphery of housing 122 for camera system 120 as further described below. Second hole 116-2, on the other hand, may be located at a farther distance from the distal end of video bougie 100 to reach a location in bougie inside space 111 that is behind camera system 120. In some embodiments, first hole 116-1 and second hole 116-2 may each be utilized for filling different parts of the inside of video bougie 100 with epoxy for one or more purposes as further detailed below.

Camera system 120 includes a housing 122, a camera 124, and a light source 126. Housing 122 includes an arm section 122-1 and a head section 122-2 as shown in FIG. 1D. Arm section 122-1 may have a cylindrical shape with a diameter that fits inside bougie inner channel 111. Head section 122-2, on the other hand, may have a shape of a hemisphere or a section of a sphere with an external diameter that matches the external diameter of distal section 114. Moreover, head section 122-2 may have a smooth surface and be formed of a material that does not harm the patient's internal passages with which it may contact, so as to be atraumatic. In some embodiments, housing 122 may be molded from plastic.

Further, camera system 120 includes an interior housing passage 123 that aligns with bougie passage111. Housing passage 123 may span the interior of both arm section 122-1 and head section 122-2. The distal section of housing passage 123, located inside head section 122-2, may include slots for housing camera 124 and light source 126. In some embodiments, these two slots may respectively have a square cross-section that fits the cross-section of camera 124 and a rectangular cross-section that fits the cross-section of LED light source 126. These slots may further provide a camera window 127-1 and a lighting window 127-2. Camera window 127-1 may have a circular shape and may provide an opening for external light to reach camera 124. Lighting window 127-2, on the other hand, may have a rectangular shape and may provide an opening for the light from light source 126 to reach outside and illuminate an area around head section 122-2. These windows 127-1 and 127-2 may be filled with an optically clear epoxy at assembly, and said epoxy may be UV-cured.

Arm section 122-1 may include a peripheral groove 125 that may span the periphery of arm section 122-1. Moreover, peripheral groove 125 may be carved at such a distance from the tip of camera system 120 that peripheral groove 125 is aligned with first hole 116-1 when camera system 120 is inserted into body part 110. This feature provides for the ability to mechanically lock camera system 120 into the body part 110 with an epoxy that fills both groove 125 and hole 116-1.

In other embodiments, the bougie does not include an opening such as the first hole 116-1. For example, FIG. 1E shows such a bougie 130 that does not include an opening in its body part for use in securing the camera system to its distal end, e.g., via epoxy. Rather, other mechanisms can be employed for attaching the camera system to the body part of bougie 130. For example, a fitting positioned at the distal end of the body part can receive the camera system and secure it in place. By way of example, in various embodiments, a barb on the outside diameter of 122-1 can be adequate to secure camera system 120 into distal body part 114's inner passage 111 and may eliminate the need for epoxy in that location.

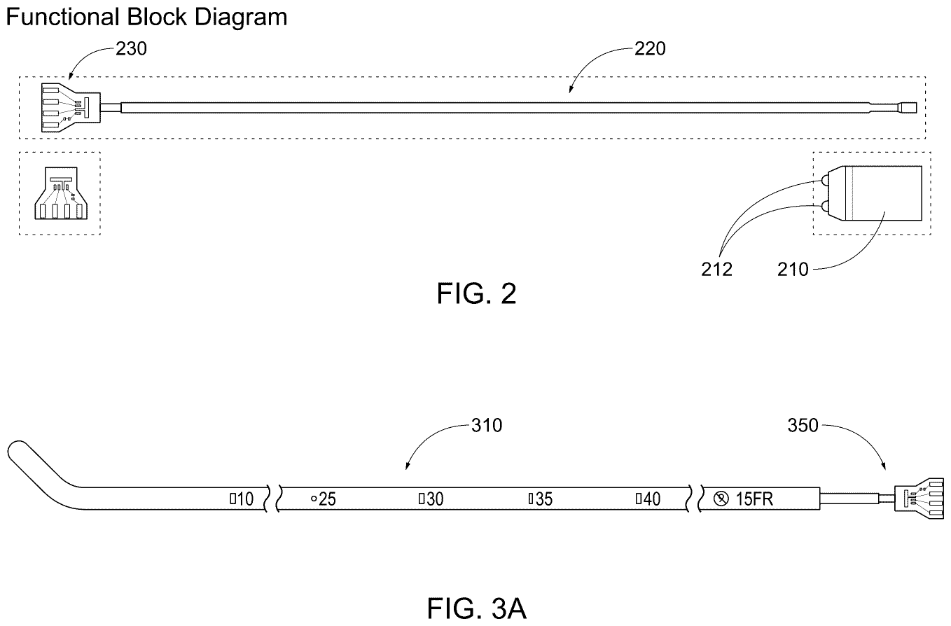

FIG. 2 shows a functional block diagram of a camera 210, camera cables 220 and camera connector 230 according to some embodiments. Camera connector 230 is configured to connect to a power source and/or a control device for the camera. Camera cables 220 may include one or more cables connecting camera connector 230 to one or more terminals 212 at the bottom of camera 210. Through terminals 212, camera 210 may receive electricity and/or control signals to operate.

In some embodiments, the cables may be custom-length as needed to extend beyond the video bougie by about 3-to-6 feet to allow the video-monitor to connect directly to the camera. In the embodiment shown in FIG. 2, camera 210 is shown to be an Omni vision camera model #OVM6946, which measures about 1.1 mm square by 5 mm long with four wires for 3.3 Volt, ground, clock, and video signal. There is a 100-ohm resistor just for the camera built into the flex-cable and a 330-ohm (approximately) resistor for current limiting the LED built into the flex-cable further downstream after the power wire bifurcates from the camera. The 2-layer flex-cable is short (˜20 mm) with 3 conductors on top and a ground layer on bottom and converts to a standard wire cable while still near the camera.

Various embodiments may utilize other combinations of other cameras and/or camera cables and connectors. For example, some embodiments may use an Omni-Vision camera model OCHTA, that may have a size around 0.65×0.65 mm. In some embodiments, use of a smaller camera may enable building a pediatric or urethral video bougie. Similarly, other embodiments may utilize an OMV6948 camera, which has a size of 1.1×1.1 mm. Yet other embodiments may utilize other cameras from other manufacturers with different costs. For example, Omnivision camera OVM7695-RACA-1B is larger (about 2.52×2.97 mm) in cross section and has about 17 wire connections but offers an all-in-one complete camera system with VGA output that could connect to a monitor via USB-C or similar connector without the need for a camera controller circuit.

A similar combination of a connector and one or more cables may connect the light source to a power source and/or a controller to the light source.

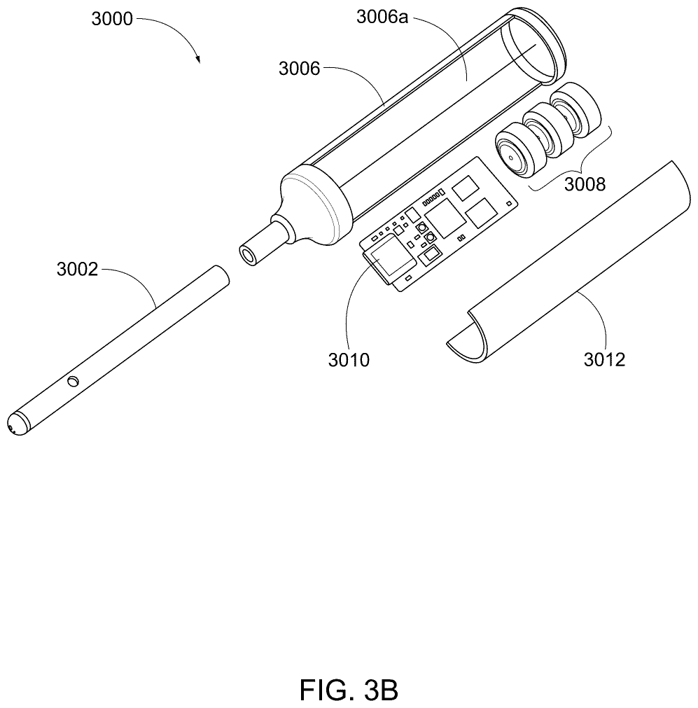

FIG. 3A shows a video bougie 310, and a cable/connector combination 350 according to some embodiments. The wire and connector are shown extending beyond the proximal end of the video bougie.

In various embodiments, the camera system including its imaging sensor and its light source can be powered by one or more batteries that are incorporated in the bougie. This advantageously provides a self-contained bougie that can be operated without a wired connection to an external power source. By way of example, such a bougie can be particularly advantageous for use in ambulances as well as in emergency cases in which time is of the essence.

FIG. 3B shows schematically a video bougie 3000 according to such an embodiment that includes an elongated tubular body part (herein also referred to as an elongated intraluminal segment) 3002 that is configured to be partially inserted into an anatomical lumen, where a camera system 3004 is positioned at a distal end of the body part 3002. Similar to the other embodiments, the camera system 3004 includes at least one light source, e.g., at least one LED, and at least one imaging sensor for generating video images. In this embodiment, the video bougie 3000 further includes a handle 3006 providing a compartment 3006a into which three batteries 3008 and a circuity 3010 can be positioned. By way of example, the circuitry 3010 can be configured to control the operation of the camera system and can be powered by the batteries. Although in this embodiment three batteries are employed, in other embodiments, the number of batteries can be different. A cover 3012 can be used to close the compartment. The batteries can provide power to the circuitry 3010 as well as to the one or more light sources (e.g., one or more LEDs) and the imaging sensor of the camera system. By way of example, and without limitation, the batteries can be 1.5 V alkaline batteries. In some embodiments, the circuitry 3010 can be wirelessly controlled, e.g., via an app residing on a mobile device, such as a mobile phone, to operate the camera system.

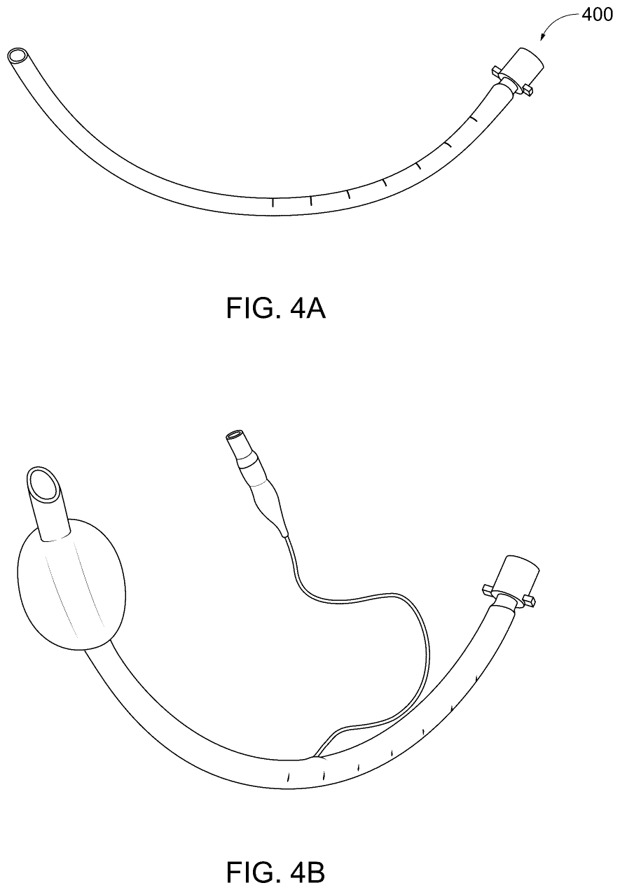

FIG. 4A shows an Endotracheal Tube (ETT) 400. In some embodiments, ETT 400 may be slipped over video bougie 100 prior to insertion into the patient. FIG. 4B shows an ETT with an inflatable cuff.

Some embodiments may utilize additional features such as an inflatable cuff, as discussed in more detail below.

In some embodiments, the following steps may be taken to assemble video bougie 100. First, the wirings for the camera and the light source may be connected to camera 124 and light source 126, respectively. Those wirings may then run through housing passage 123 and bougie passage 111 to exit video bougie 100 and connect to the respective electrical sources.

Then, camera 124 and light source 126 may be inserted into their respective recesses (slots) in head section 122-2. Afterwords, the assembled camera system 120 may be inserted into bougie passage 111. Then, epoxy may be injected through first hole 116-1 and second hole 116-2. The epoxy injected through first hole 116-1 may be utilized to secure the wirings and provide strain relief for the wirings making their connections robust if stressed by tension as the video bougie is moved forward. The epoxy injected through second hole 116-2, on the other hand, may be utilized to fill at least a portion of peripheral groove 125 and secure camera system 120 inside body part 110.

The assembled video bougie 100 may be further utilized as follows. The video bougie is inserted through an ETT, then the video bougie is guided into the patient's airway using live video imaging until it is successfully introduced into the trachea. Once the video bougie is in place as a guide, the ETT is slid (or “railroaded”) into the trachea while being rotated, allowing it to pass through the vocal cords smoothly. When the ETT reaches a measured depth, the video bougie is removed and disposed of. Inflating the ETT's cuff in the trachea keeps it in place and protects the airway from aspiration (during inhalation) in the usual way.

The above-described embodiments provide many advantages over the existing technology. To begin, the laryngoscope may not be needed, as its view is redundant. Moreover, dragging the coude (bent) tip against the tracheal rings is not needed to “feel” for correct placement. Further, the usual assistant may not be needed as the view past the epiglottis and into the larynx is now visible on a screen to which the camera may be connected. Also, the speed and accuracy of a video-bougie insertion reduces stress and trauma on the patient, thereby improving comfort, and reducing complications and misplacement. Moreover, the confirmation of ETT placement into the anatomically correct position is normally required immediately after intubation. An unrecognized incorrectly placed ETT may result in hypoxemia, and ultimately hypoxic encephalopathy or death. This extra step should no longer be necessary as the view provided by the video bougie provides placement confirmation. Further, the usual method of administering anesthesia drugs and knocking the patient unconscious may not be necessary, which means there is an opportunity to avoid the associated risks. Moreover, the risk of infection may be reduced by manufacturing the complete ETT system including the video bougie as a single-use disposable system that may be pre-packaged ETO-sterilized before shipment.

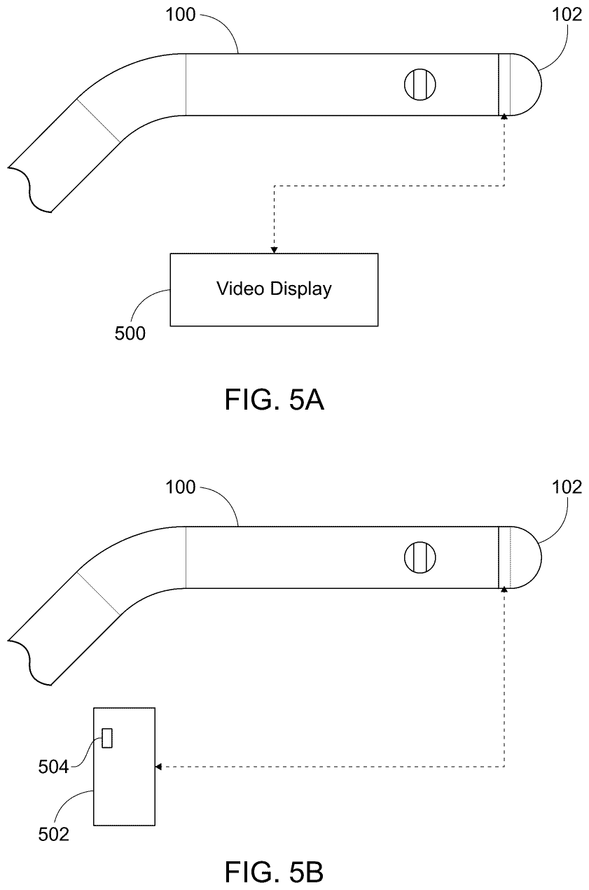

With reference to FIG. 5A, the camera system 120 of the bougie 100 can communicate, e.g., via a wired connection or wirelessly, with a video monitor 500 (herein also referred to as a video display), to transmit video images acquired by the camera system to the video display 500 for presentation to a user, e.g., a medical professional. In various embodiments, the display is not mechanically coupled to the bougie. For example, the display 500 can be positioned, e.g., as an overhead display, separately from the bougie in an operating room (OR) in which the bougie 100 is employed, e.g., to facilitate the insertion of an endotracheal tube into a patient, or to widen a narrowed anatomical lumen, etc. The separation between the bougie 100 and the display 500 provides certain advantages. For example, it can facilitate the manipulation of the bougie by a medical professional and also facilitate the viewing of video images generated by the camera system of the bougie.

Referring to FIG. 5B, in some embodiments, the camera system 102 of the bougie 100 can communicate with a mobile device 502, such as a mobile phone or tablet, via a wired or wireless connection. By way of example, the camera system can be connected to the mobile device via a wired connection to receive electrical power from the mobile device. In another example, the camera system 102 of the bougie 100 can communicate with the mobile device 502 via a wireless protocol (Bluetooth, WiFi), e.g., to transmit video images acquired via the camera system for storage and/or presentation to a user. By way of example, in some embodiments, an app 504 residing on the mobile device can allow controlling the operation of the camera system 102.

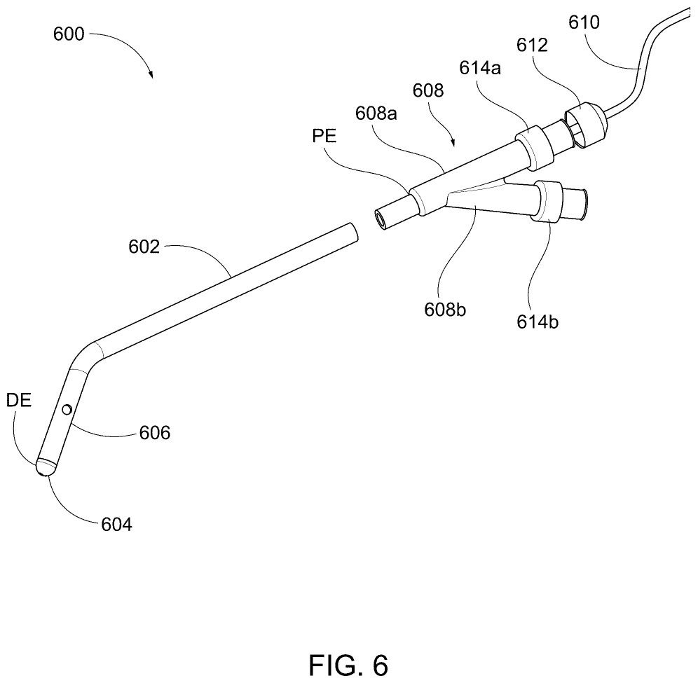

In some embodiments, a bougie according to the present teachings can include not only a camera system, including an imaging sensor and at least one light source, such as, an LED, but it can also provide a passageway for delivery of oxygen to a patient. By way of example, FIG. 6 schematically depicts such a bougie 600 that includes an elongated tubular body 602 (herein also referred to as an elongated tubular intraluminal segment) providing a hollow lumen that extends from a proximal end (PE) to a distal end (DE). Similar to the embodiments discussed above, such as the bougie 100, the bougie 600 includes a camera system 604 positioned its distal end. The camera system 604 can include an imaging sensor and one or more light sources (not visible in this figure), such as LEDs, which can be implemented, for example, in a manner discussed above with respect to the previous embodiments. However, unlike the previous embodiments, the bougie 600 includes at least one opening 606 (herein also referred to as an outlet) close to its distal end through which a flow of oxygen can be delivered to a patient once the intraluminal segment has been inserted into a body lumen of the patient.

More specifically, in this embodiment, a Y-shaped connector 608 is coupled to the proximal end of the body part 602 to provide two channels 608a and 608b, each of which is in fluid communication with the hollow lumen of the body part 602. In this embodiment, the channel 608a is employed for inserting wires 610 into the lumen of the body part 602 for supplying electrical power from an external power source (not visible in this figure) to the camera system 604, which is positioned at the distal end of the body part 602. A cap 612 can be coupled to the proximal end of the channel 608a via a luer fitting 614a to seal the proximal opening of the channel 608a.

With continued reference to FIG. 6, the channel 608b can be used for introducing a flow of oxygen into the hollow lumen of the body part 602 to be administered to a patient via its outflow through the opening 606. In this embodiment, an oxygen container (not shown in this figure) can be connected to the proximal end of the channel 608b via a tubing (not shown in this figure) that can be coupled to a luer fitting 614b to provide a flow path for oxygen from the oxygen container to the channel 608b.

In various embodiments, the ETT guide introducer may be utilized for Esophageal dilation or Urethral diagnosis (stones, stricture, etc.) and dilation (to widen the passage). Urethral bougies may be used to treat urethral strictures i.e., narrowing of urethra resulting in difficulties in urine flow. Passing a bougie into the urethra may result in widening of urethral passage. In some cases, dilation is also required prior to the passage of large instruments such as resectoscope through the urethra. In various embodiments, a male urethral bougie may be around 3 mm to 6 mm in diameter and approximately 140 mm long. Female urethral bougies, on the other hand, may only be about 70 mm long.

In various embodiments, a video bougie according to the present teachings can be employed for allowing a medical professional to widen a narrowed or obstructed anatomical passageway (such as the esophagus or the urethra) for diagnostic and/or treatment. In such applications, a bougie according to the present teachings, such as those described above, can also be utilized to guide other instruments into the passageway.

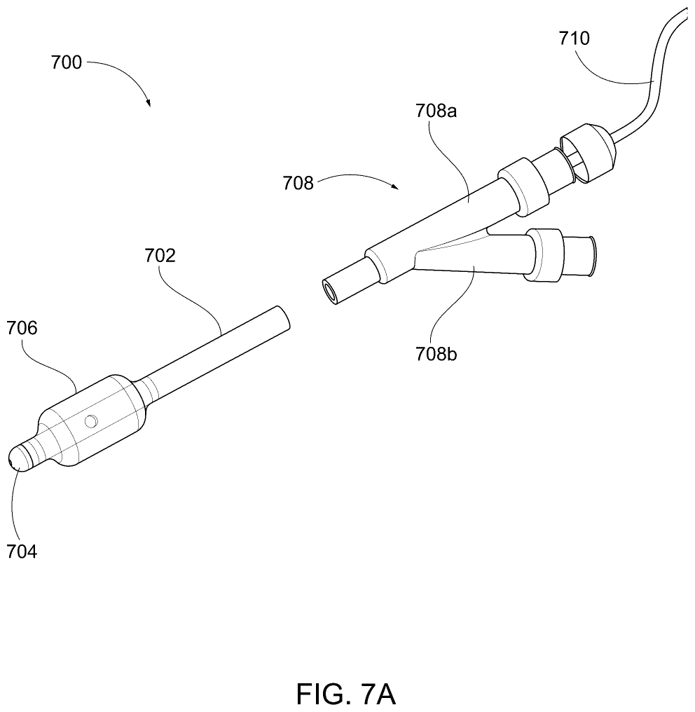

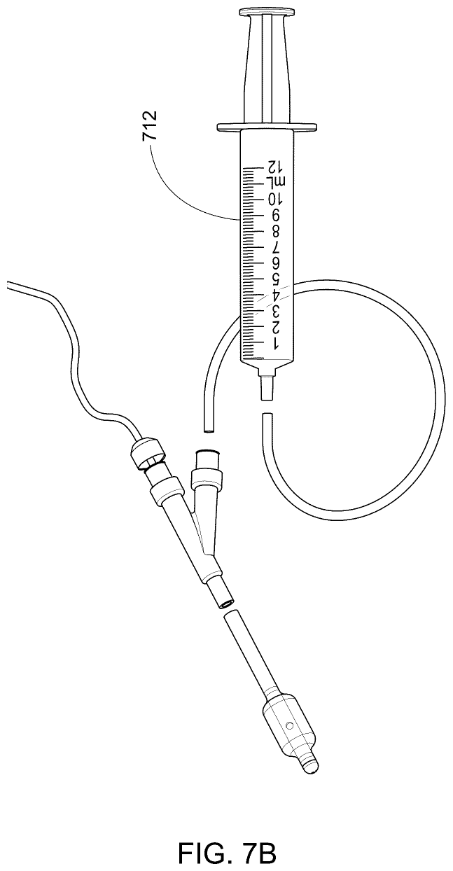

By way of example and with reference to FIGS. 7A and 7B, a bougie 700 can include a body part 702 (herein also referred to as an intraluminal segment) and a camera system 704, such as those discussed above, coupled to the distal end of the body part 702. In this embodiment, an inflatable cuff 706 (herein also referred to as a balloon) is coupled to the body part 702 upstream of the camera system 704. The balloon 706 can be transitioned from a deflated (undeployed) state to an inflated (deployed) state to help open up a narrowing of an anatomical lumen. More specifically, in this embodiment, a Y-shaped connector 708 is coupled to the proximal end of the body part 702 to provide two channels 708a and 708b, each of which is in fluid communication with the lumen of the body part. Similar to the previous embodiments, the channel 708a can be used to introduce wires 710 into the lumen of the body part 702 for providing power to the camera system 704. The channel 708b can, in turn, be used for inflating the balloon 706. For example, with reference to FIG. 7B, a syringe can be coupled to the proximal end of the channel 708b for introducing a flow of air in the lumen of the body part 702, which can exit the lumen via an opening 712 provided in the body part 702 to deliver air into the inflatable balloon thereby inflating it.

In use, the bougie 700 can be inserted into an anatomical lumen with the balloon in a deflated state and can be guided through the lumen via the video images generated by the camera system until a narrowed section of the lumen is encountered. The balloon can then be inflated in a manner discussed above to open the narrowed section.

With continued reference to FIGS. 7A and 7B, the bougie 700 has a straight profile without a bent tip, although other embodiments can provide bougies having a bent tip that also include an inflatable balloon. Further, some embodiments of a bougie according to the present teachings may include a straight body part without a balloon.

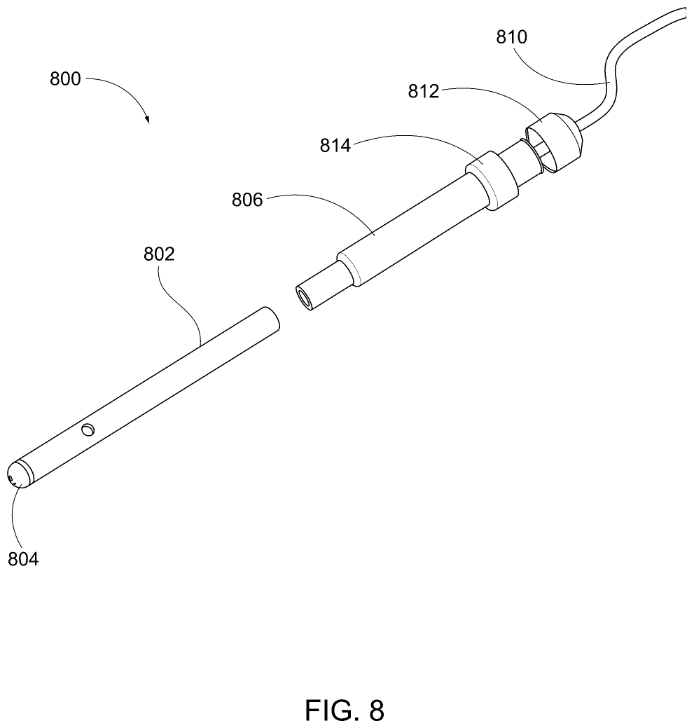

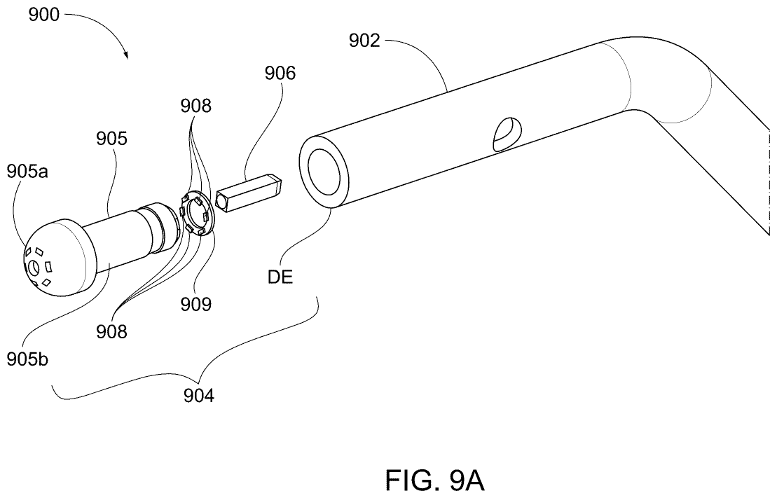

By way of example, FIG. 8 schematically depicts a bougie 800 that includes a straight, tubular body part 802 (herein also referred to as a straight, tubular intraluminal segment) that is configured to be inserted into a body lumen. Similar to the previous embodiments, a camera system 804 is coupled to a distal end of the body part 802 and a handle 806 is coupled to its proximal end to facilitate the use of the bougie. A plurality of wires 810 can be introduced via a passageway through the handle 806 into the lumen of the body part 802 to provide power to the camera system 804. A cap 812 can be used to close the proximal end of the handle via coupling with a luer fitting 814.

The straight profiles of the bougies 700 and 800 can be advantageous in providing access to and/or allowing visualization of certain anatomical lumens, such as the urethra.

As noted above, in various embodiments, a video bougie according to the present teachings is a single-use, disposable instrument, which can be discarded, including the camera system, after a single use. This advantageously eliminates the need for cumbersome sterilization procedures after each use and significantly lowers the possibility of causing cross-contamination or infections, e.g., due to an incomplete and/or incorrect sterilization protocol.

By way of example, and without limitation, in some applications, a bougie according to the present teachings can be a wire-guided bougie, which can be passed over a guidewire to allow a precise dilation in opening complex strictures, e.g., for opening esophageal strictures or blockages or for opening urethral strictures or other body lumens.

By way of example, a lubricated bougie, such as those described above, can be inserted into a body lumen, typically under local anesthesia. The bougie can then be passed through the narrowed region to gradually widen it. Further, in some cases, a balloon coupled to the distal end of the bougie can be inflated to cause widening of the stricture.

Various embodiments may utilize bougies that have various sizes such as, size 8, 10, 14, and 15 French, with or without the bent tip.

In various embodiments, a bougie according to the present teachings can include, in addition to a camera system positioned at its distal end, a light source coupled to its body part that generates light that can be detected external to the patient for providing additional data for the insertion and/or guidance of the bougie through an anatomical lumen.

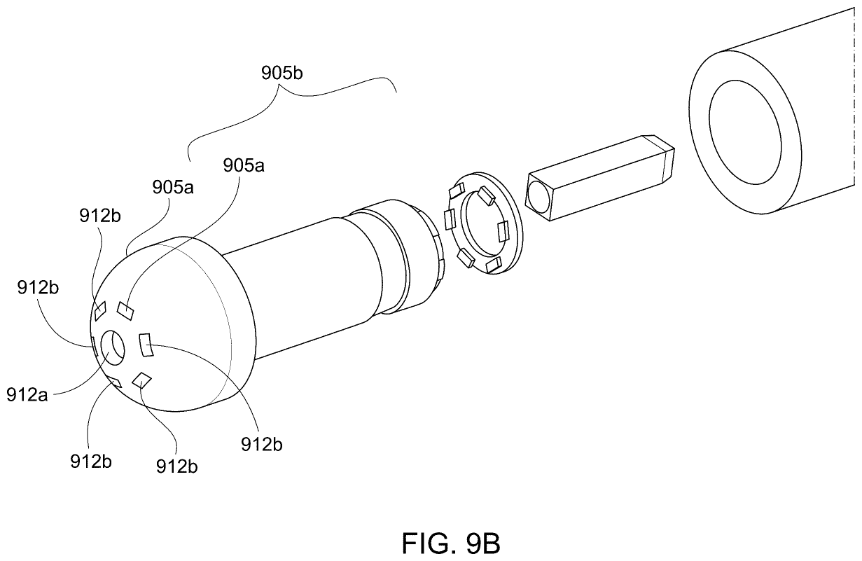

By way of example, FIGS. 9A and 9B schematically depict a bougie 900 according to an embodiment, which includes a body part (intraluminal segment) 902 having a tubular form. The body part 902 extends from a proximal end (not visible in the figure) to a distal end (DE). Similar to the previous embodiments, the body part is configured to be partially inserted into an anatomical lumen. Although in this embodiment the body part includes a bent tip, in other embodiments, the body part can have a straight profile.

A tip-mounted assembly 904 is coupled to the distal end of the body part 902 and provides a housing 905 for a video camera 906 and a plurality of LEDs 908 mounted onto a ring-shaped circuit board 909. The housing 905 includes a hemispherical head portion 905a and an arm portion 905b. The hemispherical surface of the head portion reduces the risk of injury to a patient as the body part is inserted and guided through the lumen.

The arm portion 905b includes a hollow lumen for receiving the camera 906. Further the ring-shaped PCB 909 can be coupled to a proximal end of the arm portion 905b.

With particular reference to FIG. 9B, the head portion 905a includes a central opening 912a and a plurality of openings 912b that surround the central opening 912a. The camera 906 can receive light from the external environment (e.g., a body lumen) via the central opening 912a to generate video images, which can be used, e.g., in a manner discussed above, to facilitate the insertion and/or guidance of the bougie through the lumen. The light generated by the LEDs 908 can pass through the openings 912b to illuminate the body lumen.

In this embodiment, the plurality of LEDs 908 includes three LEDs 908a, 908b, and 908c, which generate white light for primarily illuminating a body lumen into which the body part 902 is inserted in order to facilitate the acquisition of the video images. The plurality of LEDs 908 further includes three LEDs 908d, 908e, and 908f that generate red light, at least a portion of which can pass through the wall of the body lumen and its surrounding tissue to be detected external to the patient and further help with the guidance of the body part through the lumen.

By way of example, the red LEDs can emit light with a wavelength in a range of about 600 nm to about 700 nm, and preferably at a wavelength in a range of about 660 nm to about 680 nm. Further, in various embodiments, the LEDs can generate the red light at an intensity in a range of about 3 lumens to about 50 lumens. In general, the intensity of the red light emitted by the LEDs is selected to ensure that at least a portion of the emitted light can pass through the wall of the body part and the tissue surrounding a lumen into which the bougie is inserted to be detected externally.

In various embodiments, the red light emitted by the red LEDs can provide further information regarding the positioning of the body part within various anatomical lumens. By way of example, in some applications, a medical professional can use the transillumination light to determine whether a bougie has been correctly inserted into the trachea rather than the esophagus. By way of example, when the bougie is inserted into the trachea, the detection of the transilluminated light may allow a medical professional to observe distinct anatomical features, such as the tracheal rings. In contrast, if the bougie is inserted into the esophagus, the light exiting the tissue will be diffuse and can be distinguished from the light emanating from a bougie inserted into the trachea.

Although in this embodiment, three (3) white and three (3) red LEDs are employed, the number of LEDs can vary, e.g., based on a particular application.

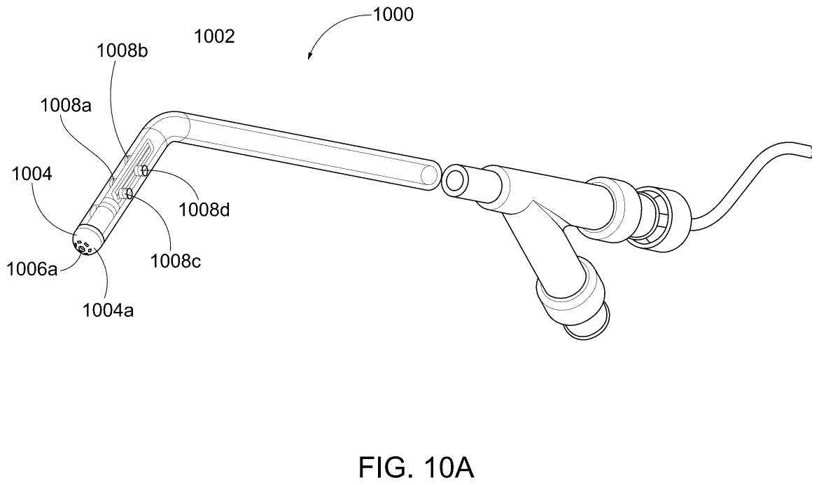

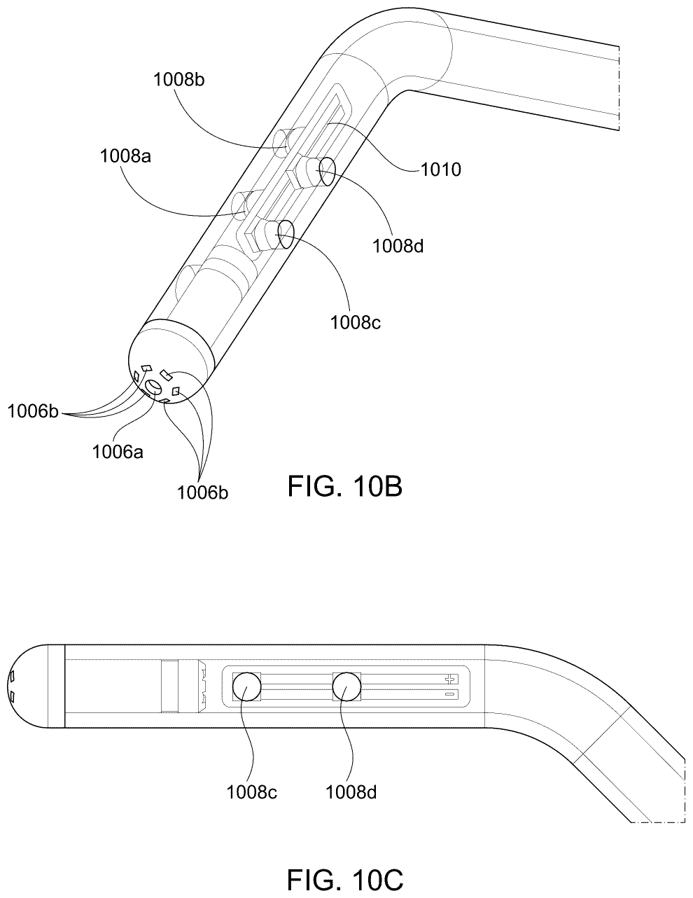

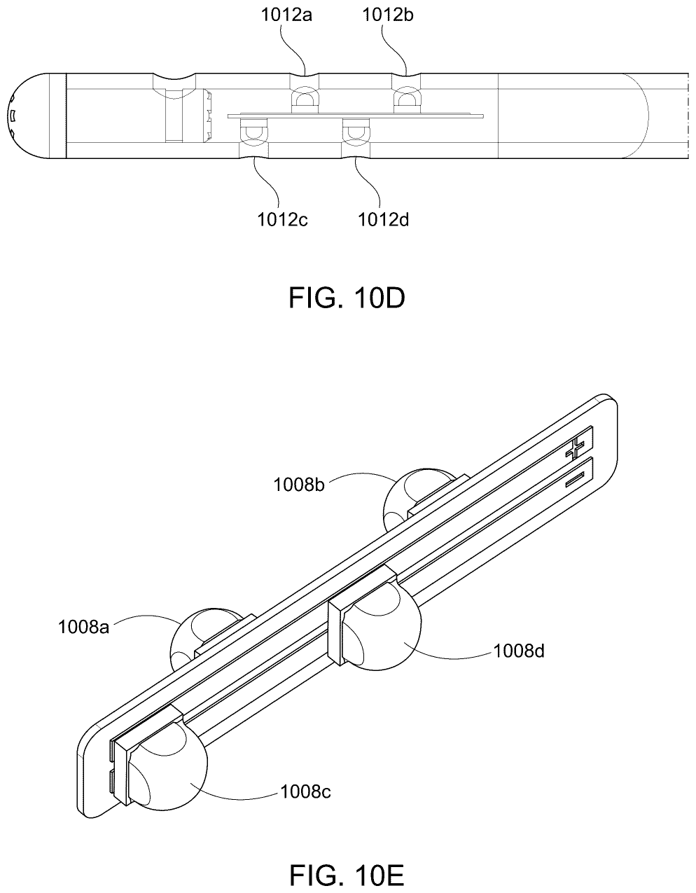

In various embodiments, the red LEDs can be incorporated into a bougie, e.g., to provide side emission of radiation that can penetrate through tissue to be detected external to a patient's body. By way of example, with reference to FIGS. 10A, 10B, 10C, 10D, and 10E, such a bougie 1000 includes a body part 1002 having a bent tip that is configured to be inserted into an anatomical lumen. The bougie 1000 further includes a tip-mounted assembly 1004, which includes a camera and a plurality of LEDs (not visible in this figure), which can be implemented in a manner discussed above. In this embodiment, the LEDs incorporated into the tip-mounted assembly emit white light for illuminating a lumen into which the body part has been inserted. Similar to the previous embodiment, the tip-mounted assembly 1004 includes a hemispherical distal surface 1004a having a central opening 1006a through which light from the external environment can reach a detector positioned in the tip-mounted assembly and a plurality of openings 1006b that surround the central opening 1006a and through which light generated by the white LEDs can exit the tip-mounted assembly.

With continued reference to FIG. 10A as well as FIGS. 10B, 10C, 10D, and 10E, the bougie 1000 further includes four red LEDs 1008a, 1008b, 1008c, and 1008d (herein collectively referred to as red LEDs 1008), which are mounted on opposed sides of a circuit board 1010, which is positioned within the lumen of the bent section of the body part. These side-mounted LEDs can be larger and therefor brighter than the tip-mounted LEDs. The red LEDs face outward so that their light can illuminate the wall of the lumen in which the body part is positioned with some of the red light passing through the tissue to be detected external to the patient's body.

More specifically, with particular reference to FIG. 10D, in this embodiment, four windows 1012a, 1012b, 1012c, and 1012d (herein collectively referred to as windows 1012), in the form of recesses made in the wall of the body part 1002 are placed in register with the red LEDs 1008a, 1008b, 1008c, and 1008e, respectively. The red light emitted by the red LEDs 1008 exits the bougie 1000 through the windows 1012. At least a portion of the red light exiting the bougie can pass through the surrounding tissue to be detected externally. By way of example, a mobile device 1014, e.g., a mobile phone, having a suitable camera can detect the red light exiting the patient's body and form an image, which can then be employed to obtain information regarding the position of the bougie within the lumen. Although in this embodiment, an external device, e.g., a mobile phone, is utilized to detect the exiting red light, in some cases, it may be possible to see the exiting red light by naked eye.

The combination of a camera at the distal end of the bougie and the red LEDs allow safe insertion and guidance of the bougie in a variety of lumens, such as the trachea, the esophagus, the urethra, among others.

It should be understood that features of one embodiment can be combined with features of another embodiment to obtain a bougie that includes all those features. By way of example, a bougie that includes an inflatable cuff (such as the above bougie 700) can also be configured in a manner discussed above to supply oxygen to a patient. For example, an opening downstream of the inflatable cuff can be employed for providing a flow of oxygen to a patient. Further, various embodiments that include one or more red lights for providing transillumination can also be configured to supply oxygen to a patient and/or include an inflatable cuff.

Those having ordinary skill will appreciate that various changes may be made to the above embodiments without departing from the scope of the disclosure.

Although some aspects have been described in the context of a system or an apparatus, it is clear that these aspects may also represent a description of the corresponding method, where a block or device corresponds to a method step or a feature of a method step. Analogously, aspects described in the context of a method step also represent a description of a corresponding block or item or feature of a corresponding apparatus.

The foregoing description of the embodiments has been presented for purposes of illustration only. It is not exhaustive and does not limit the embodiments to the precise form disclosed. While several exemplary embodiments and features are described, modifications, adaptations, and other implementations may be possible, without departing from the spirit and scope of the embodiments. Accordingly, unless explicitly stated otherwise, the descriptions relate to one or more embodiments and should not be construed to limit the embodiments as a whole. This is true regardless of whether or not the disclosure states that a feature is related to “a,” “the,” “one,” “one or more,” “some,” or “various” embodiments. As used herein, the singular forms “a,” “an,” and “the” may include the plural forms unless the context clearly dictates otherwise. Further, the term “coupled” does not exclude the presence of intermediate elements between the coupled items. Also, stating that a feature may exist indicates that the feature exists in one or more embodiments but not necessarily in all embodiments.

In this disclosure, the terms “include,” “comprise,” “contain,” and “have,” when used after a set or a system, mean an open inclusion and do not exclude addition of other, non-enumerated, members to the set or to the system. Further, unless stated otherwise or deducted otherwise from the context, the conjunction “or,” if used, is not exclusive, but is instead inclusive to mean and/or.

Moreover, if these terms are used, a set may include one or more members, and a subset of a set may include one or more than one, including all, members of the set.

The disclosed compositions, systems, methods, and apparatus are not limited to any specific aspect or feature or combinations thereof, nor do the disclosed compositions, systems, methods, and apparatus require that any one or more specific advantages be present or problems be solved. Any theories of operation are to facilitate explanation, but the disclosed compositions, systems, methods, and apparatus are not limited to such theories of operation.

Modifications and variations are possible in light of the above teachings or may be acquired from practicing the embodiments. For example, the described steps need not be performed in the same sequence discussed or with the same degree of separation. Likewise various steps may be omitted, repeated, combined, or performed in parallel, as necessary, to achieve the same or similar objectives. Similarly, the systems described need not necessarily include all parts described in the embodiments and may also include other parts not described in the embodiments. Accordingly, the embodiments are not limited to the above-described details, but instead are defined by the appended claims in light of their full scope of equivalents. Further, the present disclosure is directed toward all novel and non-obvious features and aspects of the various disclosed embodiments, alone and in various combinations and sub-combinations with one another.

While the present disclosure has been particularly described in conjunction with specific embodiments, many alternatives, modifications, and variations will be apparent in light of the foregoing description. It is therefore contemplated that the appended claims will embrace any such alternatives, modifications, and variations as falling within the true spirit and scope of the present disclosure.

What is claimed is:

1. An intraluminal navigation device, comprising:
an intraluminal segment extending from a proximal end to a distal end and configured to be inserted into a body lumen,
a tip-mounted assembly coupled to the distal end of the intraluminal segment,
said tip-mounted assembly comprising:
a surface having at least one opening,
at least one light source configured to generate illumination light to be transmitted through said at least one opening,
at least one video camera positioned to receive light entering the tip-mounted assembly through said at least one opening and configured to generate video images for guiding insertion of said intraluminal segment into said body lumen.

2. The device of claim 1, wherein said intraluminal segment comprises a recess at said distal end thereof for receiving said tip-mounted assembly.

3. The device of claim 2, wherein said tip-mounted assembly comprises:
a housing having a recess cavity in which said video camera is disposed and a second recess in which said at least one light source is disposed.

4. The device of claim 1, wherein said intraluminal segment is configured for insertion into a subject's trachea.

5. The device of claim 1, wherein said at least one light source comprises a plurality of light sources surrounding said video camera.

6. The device of claim 1, wherein said video camera is configured to transmit said video images wirelessly to a display for presentation to a user.

7. The device of claim 1, wherein said intraluminal segment includes a polymeric material.

8. The device of claim 1, wherein said device is single-use and disposable.

9. The device of claim 1, wherein said intraluminal segment is substantially tubular.

10. The device of claim 9, wherein said intraluminal segment has an outer diameter equal to or less than about 6.33 mm.

11. The device of claim 10, wherein said outer diameter of the intraluminal segment is in a range of about 4-6 mm.

12. The device of claim 1, further comprising a compartment for housing one or more batteries and/or circuitry.

13. The device of claim 1, wherein said intraluminal segment has a hollow lumen.

14. The device of claim 13, wherein said intraluminal segment includes one or more openings.

15. The device of claim 14, further comprising:
an inflatable cuff coupled to said intraluminal segment upstream of said camera system and in fluid communication with said one of the openings in said intraluminal segment, and
a mechanism coupled to the proximal end of the intraluminal segment for inflating the inflatable cuff via said hollow lumen and said opening in said intraluminal segment.

16. The device of claim 1, wherein said tip-mounted assembly further comprises at least one red light source generating red light and positioned such that the red light is transmitted through said opening in the intraluminal segment with at least a portion of the transmitted red light passing through a wall of said body lumen and surrounding tissue so as to be externally detectable.
